(19) United States
(12) Patent Application Publication
(10) Pub. No.: US 2025/0152000 A1
(43) Pub. Date: May 15, 2025

(54) WIDE-FIELD SWEPT-SOURCE OCT AND METHOD FOR MOVING OBJECTS

(71) Applicant: Carl Zeiss Meditec AG, Jena (DE)

(72) Inventors: Daniel BUBLITZ, Rausdorf (DE); Dirk SEIDEL, Jena (DE)

(21) Appl. No.: 18/709,202

(22) PCT Filed: Nov. 8, 2022
(86) PCT No.: PCT/EP2022/081158

§ 371 Date: Nov. 19, 2024

(30) Foreign Application Priority Data
Nov. 12, 2021 (DE) 10 2021 129555.6

(51) Int. Cl. A61B 3/10

(52) U.S. Cl. CPC A61B 3/102

(57) ABSTRACT

A wide-field swept-source OCT method that images a moving object including an anterior chamber. Including providing wavelength-tuned illumination radiation in individual illumination pulses of different centroid wavelengths, illuminating the object and imaging the object on a 2-D detector having an image recording cycle of exposure intervals and read-out intervals, emitting the pulses as a series of first pulses and second pulses, with pulses with the same centroid wavelengths repeating at least once over the illumination pulse pairs, synchronizing the pulses and the detector such that the illumination pulse pairs are grouped around every second of the read-out intervals of the sequence, and generating image pairs from the detector signals corresponding to the illumination pulse pairs, determining changes between the image data of the illumination pulses with the same centroid wavelength repeated over the illumination pulse pairs and using the changes to correct movements of the object in the image data.

The invention relates to a wide-field swept source OCT and method for moving objects, in particular the anterior chamber of the human eye.

Ophthalmological OCT systems have been used for many years to measure the human retina and also structures in the anterior chamber of the eye. A known anterior chamber OCT system was developed by Carl Zeiss in the form of a confocal scanner.

For anterior chamber measurements, OCT systems, which have been developed for retinal imaging, are often adapted by means of an attachment optical unit. Since the imaging properties for anterior chamber measurement are different from those for retinal measurement and since the OCT systems are optimized for the retina, the imaging performance in the anterior chamber is not optimal. The essential differences when imaging these two tissues are the following:

1. All beams that reach the retina are cut off by the iris, so that OCT systems intended for the retina all operate with a similar pupil size of 1.0 to 1.3 mm and resulting numerical apertures of a maximum of 0.04. As a result, the lateral resolution is limited, but the depth of field, which determines the accessible depth range during measurement, remains sufficient for a visual representation of the retina up to the measurement depth of just under one millimeter, which is limited by the scattering.

2. The tissues in the retina, especially the retinal pigment epithelium and the choroid behind it, are highly scattering. To minimize the resulting multiple scattering, most OCT systems are designed as fully confocal systems. Since the OCT illumination and detection waves are imaged by the human eye lens, which has certain aberrations, only slightly better lateral resolution can be achieved even in very small image fields and depth ranges.

3. The tissues in the anterior chamber (cornea, aqueous humor, iris and eye lens) are usually much less scattering, and so non-confocal methods could also be used. Since the penetration depth of the light wave fields into the eye is also only low, aberrations caused thereby are also significantly less problematic, so that some anterior chamber systems can be used with numerical apertures of over 0.2 and resolutions up to the μm range. The resulting depth of field for the various applications is between a few micrometers and a few 10 micrometers. The depth range to be visualized in the anterior chamber is significantly larger and is approximately 6 mm in the tissue (depending on the exact application).

Therefore, there are basically two optimizations for confocal anterior chamber recordings, a high-resolution confocal scanner that sequentially measures the anterior chamber in a great many depth planes of approximately one depth of field each and combines the images. Alternatively, the numerical aperture nA can be reduced until a depth of field of approximately 8 mm in air is achieved, which corresponds to a nA=0.007 at 840 nm and a resulting lateral resolution of approximately 73 μm at 840 nm.

In the prior art, non-confocal wide-field OCT systems which are realized with fully coherent illumination are also known. Since these systems are fully coherent and at the same time non-confocal, the detected waves can be computationally propagated/focused retrospectively into each sample plane and thus the accessible depth range can be partially decoupled from the lateral resolution. Such methods and coherence tomography devices are known from DE 10 2014 115 157 A1. Similar approaches can also be found in DE 10 2014 115 153 A1, DE 10 2014 115 155 A1, DE 10 2015 101 251 A1 and WO 2017/137567 A1.

In DE 10 2018 13 0396 A1, a method of optical coherence tomography is described, which is illuminated with a scattered, fully coherent illumination wave. There are also special embodiments for a wide-field image recording with a 2D spatially resolved sensor. The described method is preferably used for volume imaging of biological tissue, such as the human eye. Moreover, it could also be used on technical samples, however.

The illumination of a holoscopic system with a scattered, but fully coherent illumination wave offers two key advantages. Due to the scattered wave, significantly higher illumination intensities can be radiated without risking damage to the eye. This can increase the recording speed because more light is available. This advantage does not occur as strongly in anterior chamber systems, since the component of the radiation that passes through the iris and reaches the retina can also be influenced here by an adapted illumination wave. In addition, such systems can also be illuminated in principle with wavelengths in the range of 1.3 μm to 1.55 μm, which could not transmit to the retina due to the high water absorption of the vitreous body.

The second advantage of the scattering wave illumination that should be mentioned is that repeatedly scattered interference light components can be differentiated from signal light components which are scattered once, and thus the contrast of the recordings in highly scattering tissues can be improved. This advantage is also hardly noticeable in the less scattering tissues of the anterior chamber.

Depending on the size of the image field to be measured and the lateral resolutions to be achieved, very large pixel resolutions may result which can no longer be achieved with available fast camera sensors. In these cases, the image field is recorded in a plurality of partial images, which are computed/combined to form an overall image. Due to shadowing effects at the boundaries of the partial image fields, the partial images must be recorded with a certain overlap, which is determined by the ratio of the depth range to be measured and the depth of field. In order to achieve an overall very efficient measurement under these circumstances, it is particularly preferable to compose the overall image from as few partial fields as possible which are as large as possible.

Recording the different wavelengths with a 2D camera for computing the depth function requires coherent, i.e. phase-fixed recording. Residual movements of the eye, when working without contact glass as applicatively preferred, limit the time for which this phase correlation can be maintained to about 10 to 25 ms. There are methods (e.g. EP 3 517 021 A1) in the prior art that can estimate movements occurring in retinal recordings from the partial images even over periods of less than one second and can correct them numerically during evaluation. In contrast to retinal recordings, anterior chamber images are less strongly spatially structured, however, which means that these approaches cannot be transferred safely.

If the intention is to merely measure the cornea in high resolution, wavelength tuning over at least about 50 different wavelength images is needed when working with a curved zero-delay surface by way of suitable illumination. If the intention is to represent the entire anterior chamber, wavelength tuning over about 400 wavelength images, all of which would have to be recorded within about 25 ms, is required.

For these frame rate requirements, very expensive high-speed cameras such as the PhotronSAZ camera, which achieve frame rates in the range of up to 20,000 fps at about 1 Mpixel resolution, are suitable. However, these camera systems are not available with sufficiently good pixel resolutions and are therefore once again not suitable overall.

The invention is therefore based on the object of providing an accelerated fully coherent wide-field OCT system, wherein the camera sensor operates with high resolution (i.e. pixel number), but does not have to reach speeds above 1000 fps. In particular, the speed of the system should be suitable for measurements of the anterior chamber of the human eye without contact glass.

The invention is defined in the independent claims. The dependent claims relate to preferred developments.

The wide-field swept source OCT and the method are used to image a moving object, in particular the anterior chamber of the human eye. The method comprises the following steps:
providing illumination radiation which is tuned in the wavelength and comprises individual illumination pulses of different centroid wavelength. The illumination pulses are emitted as a series of illumination pulse pairs, each consisting of a first illumination pulse and a second illumination pulse. In the illumination pulse pairs, the centroid wavelength of the first illumination pulse differs from the centroid wavelength of the second illumination pulse. In a plurality of the illumination pulse pairs, a centroid wavelength of one of the illumination pulses of a preceding one of the illumination pulse pairs is repeated.
illuminating the object with the illumination radiation, wherein the illumination radiation is scattered or reflected back in the object as measurement radiation, and imaging the measurement radiation coming from the illuminated object onto a 2D detector. Illumination is preferably realized by coherent wide-field illumination.
operating the detector according to an image recording cycle comprising a sequence of exposure intervals and readout intervals.
synchronizing the illumination pulses and the sequence of exposure intervals and readout intervals in such a way that the illumination pulse pairs are grouped around every second one of the readout intervals of the sequence and, for each illumination pulse pair, the first illumination pulse is emitted during a last third of one of the exposure intervals and the second illumination pulse is emitted during a first third of the next of the exposure intervals.
reading image data from the detector for each exposure interval and assigning the image data to the centroid wavelengths of the illumination pulse emitted in the respective exposure interval. Image pairs are generated according to the illumination pulse pairs.
determining changes between the image data of the illumination pulses having the same centroid wavelength, which are repeated across the illumination pulse pairs, and evaluating the image data and using the changes to correct movements of the object in the image data.

The wide-field swept source OCT is designed to implement the method accordingly. It comprises:
a radiation source configured to emit the illumination radiation, which has been tuned in the wavelength and comprises the individual illumination pulses of different centroid wavelength, wherein the illumination pulses are emitted according to the method as a series of illumination pulse pairs having the centroid wavelengths explained above,
the 2D detector, also referred to here as the camera, which executes the image recording cycle comprising the sequence of exposure intervals and readout intervals,
a beam path for illuminating the object with the illumination radiation and for imaging the illuminated object onto the 2D detector. The illumination preferably is effected in the form of coherent wide-field illumination.
a control device that implements the method. It controls the 2D detector and the radiation source and synchronizes them as mentioned regarding the method. It thus generates the aforementioned image pairs, determines changes between the image data of the illumination pulses having the same centroid wavelength, which are repeated across the illumination pulse pairs, and uses them for referencing when evaluating the image data and thus corrects movements of the object in the image data.

The method will be explained here by means of an anterior chamber recording, but the description applies to all medical and technical wide-field SS-OCT systems where object movements limit coherent recording. In particular, the presented solution is also suitable for volume imaging of scattering media for microscopic in-vitro applications. Furthermore, aspects described for the method apply in the same way to the OCT and vice versa.

Directly successive illumination pulse pairs are spaced apart from one another by a multiple of the distance between the two illumination pulses in the illumination pulse pair. Preferably, the distance between successive illumination pulse pairs is at least the duration of a sequence of exposure interval and readout interval, preferably at least 1⅓ times, particularly preferably at least 1.5 times the duration. The distance is in each case defined by the end of the previous illumination pulse and the beginning of the next illumination pulse.

Each illumination pulse pair is assigned an image pair, hereinafter also referred to as a double image, consisting of two single images, each of which is assigned the centroid wavelength of the corresponding illumination pulse of the illumination pulse pair. In a plurality of illumination pulse pairs, a centroid wavelength of one of the illumination pulses of a preceding one of the illumination pulse pairs is repeated. This defined structure allows movement correction across the image pairs, because for exactly one single image of each image pair, there is a single image in at least one other image pair that is assigned the same centroid wavelength. An object movement for these image pairs to which the same centroid wavelength is assigned is thus determined from a comparison of the single images and corrected for the evaluation of the other two single images of the image pairs.

This correction is particularly simple when exactly one centroid wavelength of one of the illumination pulses of the immediately preceding one of the illumination pulse pairs is repeated in a plurality of illumination pulse pairs.

For example, this creates illumination pulse pairs (and thus image pairs) with a wavelength sequence (lambda-0, lambda-1), (lambda-1, lambda-2), (lambda-2, lambda-3), etc. In general, the elements of the sequence of illumination pulse pairs then satisfy the recursive definition (lambda-k, lambda-k+1) with a running index k from 1 to n. Of course, the same centroid wavelength can also always be repeated as the reference centroid wavelength. This provides the recursive definition (lambda-0, lambda-k) with a running index k from 1 to n and lambda-0 as the reference centroid wavelength. Both variants (lambda-0, lambda-k) and (lambda-k, lambda-k+1) are mathematically equivalent. Technically, the first variant is preferable, because it requires only one tunable laser. Since the lambda-0 can then also have a greater wavelength distance from all other wavelengths lambda-k, efficient dichroic combining is also possible in 2-laser systems, which is excluded for the second variant, since the wavelengths sometimes differ only very little.

In principle, arbitrary distributions of the centroid wavelengths in the illumination pulse pairs are possible, as long as a correction of a single image is possible for each image pair by comparing the other single image with a single image of another image pair and thus a direct or indirect reference to another, already movement-corrected or movement-correctable single image is possible. Thus a sequence (lambda-0, lambda-1), (lambda-2, lambda-3), (lambda-4, lambda-5), (lambda-6, lambda-7), (lambda-1, lambda-2), (lambda-3, lambda-5), (lambda-4, lambda-7) would likewise be suitable.

The wide-field SS-OCT is preferably realized as a fully coherent system. These are also referred to as holoscopy systems. The system is particularly preferably realized with an off-axis detection arrangement, as this makes it possible to suppress the excess noise and also the DC component and the largest part of the autocorrelation signal during evaluation. For this purpose, the light scattered back by the sample is superimposed with the reference wave at an off-axis angle, made to interfere and detected. It is important here that the camera sensor realizes sampling that is 2 to 3 times higher in the direction of the off-axis angle in order to be able to resolve all spatial frequencies of the interference with the reference wave. Use of 3-pixel off-axis detection is particularly preferred. The reference wave comprises phase deviations of approximately 120° per pixel in the off-axis direction here.

A camera with a pixel resolution of well over 1 Mpixels, which is preferably technically designed as a global shutter camera, is preferably used. This type of readout makes it possible to record two images in a very short time, which is important for the method described here. However, there are also rolling shutter cameras that achieve a sufficiently fast detection of well over 50 fps with a sufficient number of pixels and can be used for the type of detection described.

For the method, the illumination can be conventionally effected with a fully coherent spatial single-mode wave or alternatively with a scattered fully coherent multimode wave, as described in DE 2018 130 396 A1.

Microscopic full field OCT systems, such as those described in C. Apelian et al., Biomedical Optics Express Vol. 7, No. 4, 2016, “Dynamic full field optical coherence tomography: subcellular metabolic contrast revealed in tissues by interferometric signals temporal analysis,” may be mentioned as an application for very fast volume imaging with microscopic resolution over measurement depths of many depths of field. Since multiple scattering constitutes a very significant problem for these microscope systems, scattering wave illumination and an evaluation which effectively suppresses multiple scattering are particularly preferred for these principles.

It goes without saying that the features mentioned above and the features yet to be explained hereinafter can be used not only in the specified combinations but also in other combinations or on their own, without departing from the scope of the present invention.

The invention will be explained in even greater detail below on the basis of exemplary embodiments with reference to the accompanying drawings, which likewise disclose features essential to the invention. These exemplary embodiments are used for illustration only and should not be construed as limiting. For example, a description of an exemplary embodiment having a multiplicity of elements or components should not be construed as meaning that all of these elements or components are necessary for implementation. Rather, other exemplary embodiments may also contain alternative elements and components, fewer elements or components, or additional elements or components. Elements or components of different exemplary embodiments can be combined with one another, unless indicated otherwise. Modifications and variations that are described for one of the exemplary embodiments can also be applicable to other exemplary embodiments. In order to avoid repetition, elements that are the same or correspond to one another in different figures are denoted by the same reference signs and are not explained repeatedly. In the figures:

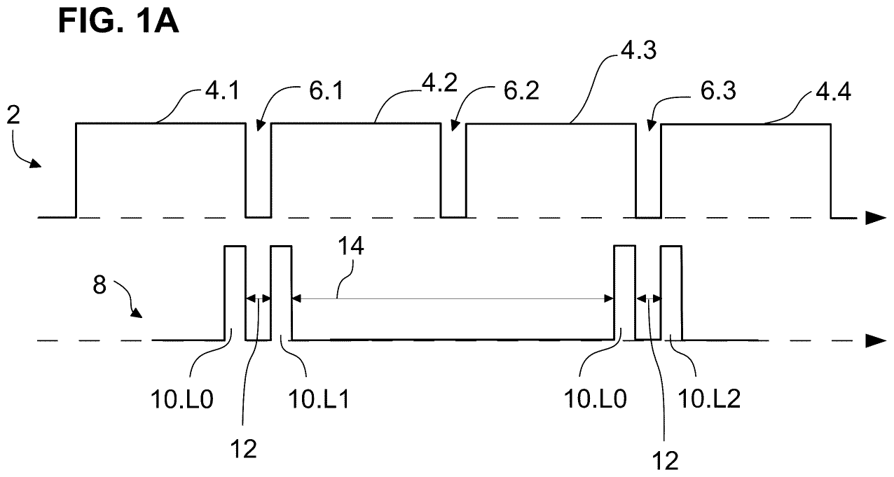

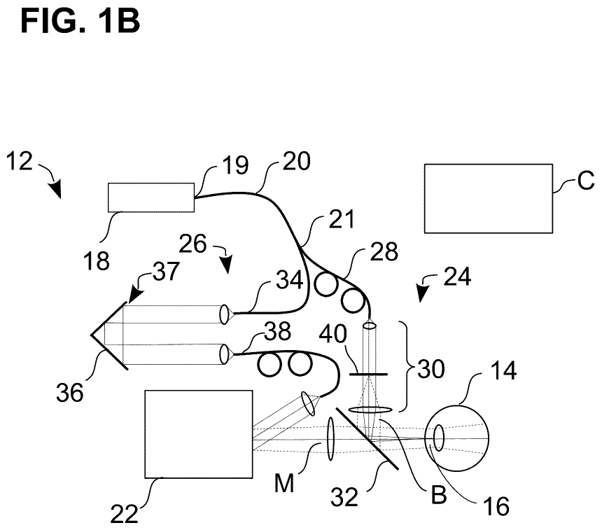

FIG. 1A shows a sequence schedule for a method for OCT imaging,

FIG. 1B shows a block diagram for an OCT apparatus,

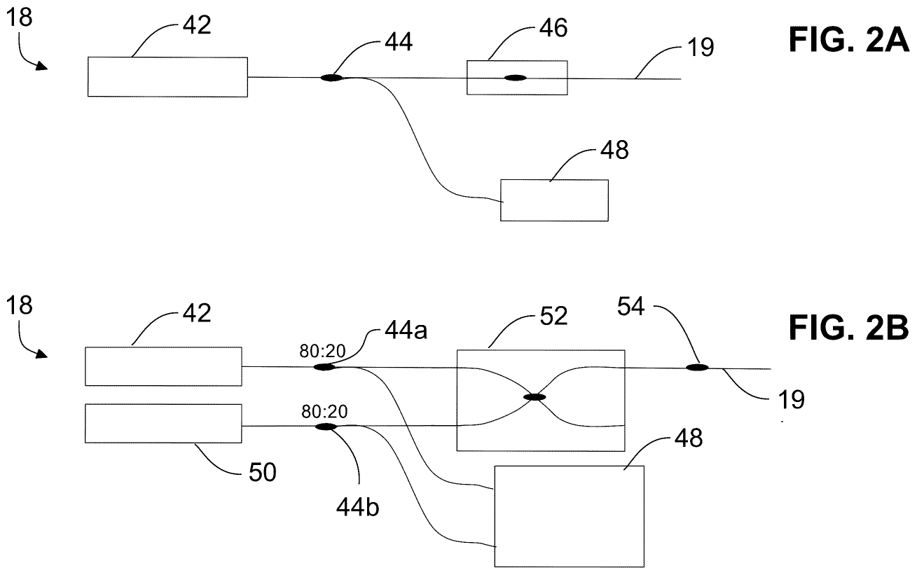

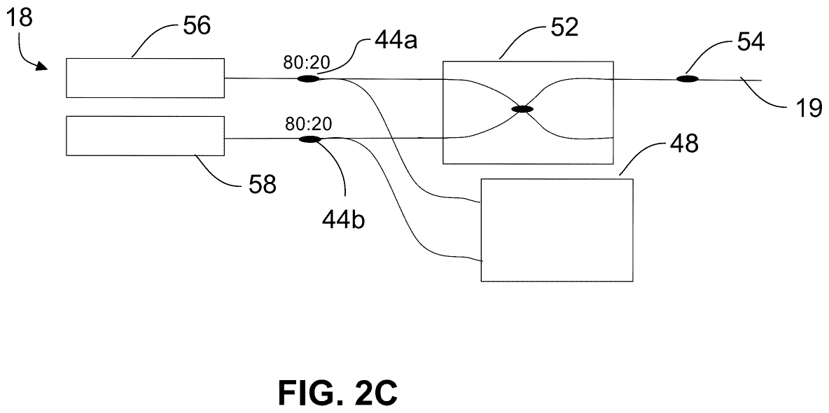

FIG. 2A-2C show possible light sources for the apparatus of FIG. 1B,

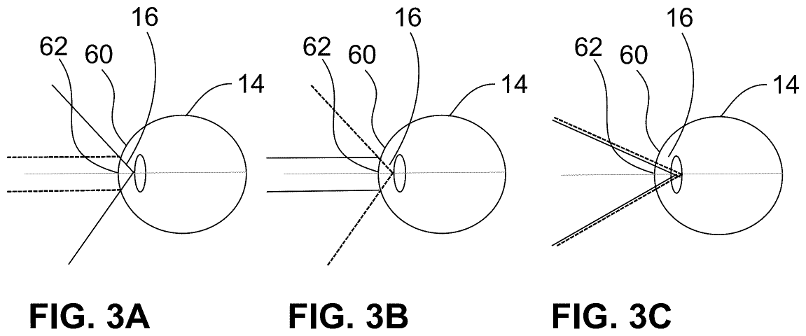

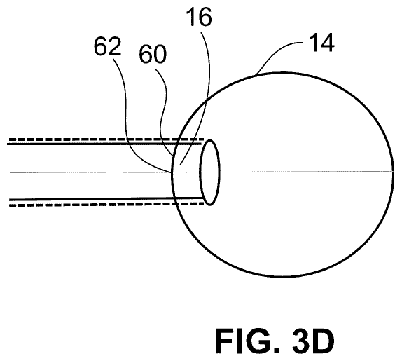

FIG. 3A-3D show possible beam geometries for illumination in the apparatus of FIG. 1B.

For fast SS-OCT recording with an image sensor whose frame rate, i.e. minimum exposure time, is actually too slow for the desired imaging speed, the diagram of the exposure and readout of the detector, which is schematically shown in FIG. 1, is used. FIG. 1A shows a sequence of the exposure intervals in a conventional camera, which is designed here, for example, as a global shutter sensor. In sequence 2, exposure intervals 4.1, 4.2, etc. follow one another. Each exposure interval represents an integration interval over which the pixels of the sensor collect electrical charges as long as they are illuminated. The duration of the exposure interval 4.1, 4.2, 4.3, etc. determines the frame rate. After the individual exposure intervals 4.1, 4.2, 4.3, etc. have ended, the sensor is read in each case over a readout interval 6.1, 6.2, etc., i.e. the charges that have accumulated in the pixels are transferred to a readout register. Then the next integration, i.e. the next exposure interval, can begin. In particular, this means that the sensor collects charges in its pixels during the exposure interval 4.1 and transmits them to the readout register during the readout interval 6.1, and no charges can accumulate during this time. Then a next exposure interval 4.2 begins, in which charges in the pixels are collected again. They are then transferred back to the readout register in the readout interval 6.2 so that the next exposure interval 4.3 can begin. The duration of the exposure intervals is such that the movement-related problems on the sample, for example on the eye in the case of an ophthalmological anterior chamber OCT, would thus no longer be tolerable. Especially for the preferred holoscopy, it would be the case that the required phase rigidity between the individual recordings during tuning of the laser would no longer be present.

Therefore, the exposure, that is, the illumination of the sample, is effected in a way as is illustrated in the sequence 8 in FIG. 1A. At the end of the first exposure interval 4.1, a short illumination pulse 10.L0 is radiated in, i.e. only during this time is light incident on the pixels in the exposure interval 4.1. At the beginning of the subsequent exposure interval 4.2, an illumination pulse 10.L1 is emitted, which is at a different wavelength according to the swept-source principle of the SS-OCT. It is comparatively short relative to the exposure interval 4.2, so that it substantially collects only sample light due to the illumination pulse 10.L1. The distance 12 between the two illumination pulses 10.L0 and 10.L1 is illustrated by the double-headed arrow and is substantially limited only by the duration of the readout time 6.1. The distance 12 is so small that no interference occurs during this time, i.e. the required phase rigidity between the image collected during the exposure interval 4.1 by the exposure pulse 10.L0, and the image of the exposure interval 4.2, which originates from the illumination pulse 10.L1, is present.

This synchronized sequence is now repeated, wherein the illumination pulse 10.L0, which is radiated in toward the end of the exposure interval 4.3, is now followed by an illumination pulse 10.L2, which has a different wavelength than the illumination pulse 10L. 1. The temporal distance 14 between the illumination pulse 10.L1 and the next pulse is so large that the required phase rigidity would no longer be present here. However, since phase rigidity is present again between the successively incoming illumination pulses 10.L0 and 10.L2, a phase correction over the images at the illumination pulse 10.L1 (recorded during the exposure interval 4.2) and the illumination pulse 10.L2 (recorded during the exposure interval 10.3) can be carried out without any problems.

Thus, the inherently insufficient frame rate of the image sensor is compensated in this way, since double images are generated. These double images in each case correspond to an illumination pulse at a reference wavelength, in FIG. 1A the illumination pulse 10.L0, wherein “L0” symbolizes the reference wavelength lamda-0, and to an illumination pulse with a wavelength that changes according to the wavelength tuning duration, symbolized in FIG. 1A by the addition “L1” or “L2”.

An S-65A70 camera from Adimec, Eindhoven, NL is suitable for the execution. This global shutter sensor has a resolution of 65 Mpixels and can be read at 70 fps. The sensor is set to the longest possible exposure time of 14 ms, which still achieves the full frame rate. After 14 ms, the charges that have accumulated in the pixels are transferred to a readout register within less than 50 μs (frame overhead time), and the integration of the next frame is started. During the integration of the next frame, the first frame is simultaneously digitized and transferred to the evaluation unit.

If this scheme is appropriately synchronized with the pulsed illumination for the entire camera, for example such that the illumination is activated only for the last millisecond of the first integration interval 4.1 and for the first millisecond of the second integration interval 4.2, two fully resolved camera images are recorded in a total of less than 2.05 ms. No significant movement artifacts occur in such short periods of time.

The pulsed mode of operation enables measurement speeds to be achieved that are significantly higher than the nominal frame rates of the camera. In addition, the laser limits for permissible peak powers can be further increased.

In the SS-OCT system, a defined number of images are recorded at the illumination pulses 10.L1, 10.L2, etc. under illumination using an SS laser while the laser is tuned linearly and continuously in its k-number (k=2 pi/lambda). The tuning speed is adapted to the number of images to be recorded that is required for the depth range and to the frame rate of the camera.

For the SS-OCT approach, the tuning rate of the laser is therefore chosen to be less than or equal to the frame rate of the camera. The frame rate of the camera, the incoming radiation of the illumination pulses is adapted according to FIG. 1A to the tuning rate of the camera such that double images for example according to (lambda-0, lambda-1), (lambda-0, lambda-2), (lambda-0, lambda-3), . . . (lambda-0, lambda-n) are recorded. Each double image is fully coherent in itself due to the short recording time, but the double images among themselves may comprise movement artifacts due to the distance 14, since the distance 14 does not meet the condition of phase rigidity, which is essential for the evaluation. However, since for each double image there is again a recording with the reference wavelength lambda-0, the phase position within the double images is fixed and can thus be indirectly also adjusted to one another across the double images. Thus, in the i-th double image, the differential phase between the images at lambda-0 and at lambda-i is determined and used for further evaluation. In this way, the phase errors caused by movement artifacts are compensated and the recordings for lambda-0 to lambda-i are coherent with respect to one another again in terms of evaluation.

This applies to axial movement artifacts. Lateral movement artifacts are optionally likewise corrected by cross-correlating the reference images with lambda-0 with one another. As a result, the shift vector between the recordings is obtained and the lambda-0 images and the associated lambda-i images can thus be compensated for in terms of their shift.

Of course, the phase referencing does not have to be done with the wavelength lambda-0, but can be done with any wavelength from the wavelength sequence or even with an additional wavelength, which is provided, for example, by a fixed frequency reference laser.

If the camera achieves high frame rates, it may also be sufficient to perform a reference measurement with the fixed wavelength only every 3, 10 or more wavelength images. In general, the reference measurement is performed every m images (m being a natural number greater than zero).

The distances between the reference images of the same frequency can also be selected to be so large that measurable movement artifacts/phase shifts occur. In this case, it is particularly preferred to stabilize the phase positions of the reference images 2 pi (unwrapping), to interpolate the phase changes for all intermediate times and to use them to correct the different wavelength images.

FIG. 1B schematically shows an October 12, which takes three-dimensional images of an eye 14, specifically in particular of the anterior chamber 16. The described embodiment is described using the example of a fiber-based swept-source system, but can also be transferred similarly to free-beam structures. Source radiation from a radiation source 18 which is tunable with regard to its wavelengths, for example a corresponding laser, as it will be explained below by way of example with reference to FIG. 2-4, is coupled from an output 19 of the radiation source 18 into a fiber 20. The source radiation lies, for example, in the infrared wavelength range, wherein this wavelength range is also referred to as “light” in this description. This term subsumes all radiation of the electromagnetic spectrum which complies with the laws of optics. The eye 14 is imaged by optical units (not further designated) onto a detector 22, which has the readout scheme explained with reference to FIG. 1A according to the sequence 2.

According to the fiber-based design, the fiber 20 merges into a splitter 21, which divides the source radiation into a measurement arm 24 and a reference arm 26. A fiber 28 is connected to the splitter 21 in the measurement arm 24, and the illumination radiation B emerging at the fiber end is modified by means of an illumination optical unit 30 with regard to the illumination modes—in particular, a diffusing plate 40 is provided for this purpose, the effect of which is described, for example, in DE 10 2018 130 396 A1—and then guided to a beam splitter 32. From there, it reaches the anterior chamber 16 via a front optical unit (not drawn).

This illumination radiation is scattered back in the anterior chamber 16 from different depths z within a depth of field range. The backscattered radiation is collected as measurement radiation M and reaches the detector 22. The beam splitter 32 thus separates the measurement radiation M from the illumination radiation B. The detector 22 has a spatial resolution, i.e. it allows a resolution of the intensity distribution over the beam cross section according to the holoscopy principle. The detector 22 is preferably conjugate to the eye pupil. However, since the lightwave field, if it is known in a plane with absolute value and phase, can be calculated for any other plane by way of numerical propagation, the detection may also be implemented in other planes, e.g., for a plane in the anterior chamber 16.

The radiation separated by the splitter 21 into the reference beam path 26 enters a fiber 34 and is radiated obliquely onto the detector 22 via a deflection mirror 36 and a further fiber 38 with a settable path length. This results in a superposition of reference radiation and measurement radiation M, here by means of what is known as off-axis detection. The path length adaptation 37 is realized in the illustrated exemplary embodiment as a free-beam path. This is likewise optional, as is the use of the mirror 36. The prior art discloses various measures to adjust the optical path length of a beam.

For carrying out the image generation, the October 12 comprises a control device C, which synchronizes the wavelength tuning of the radiation source 18 and the operation of the detector 22 according to the principles explained with reference to FIG. 1A, uses in each case the referencing of the phase relationship from the double images to compensate for the distance 14, and generates corresponding holoscopic images. The latter is known in the prior art.

The laser cannot be modulated during the measurement for fundamental physical reasons in order to achieve a degree of temporal coherence necessary for the measurement. Therefore, the emitted light of the laser is amplified, if necessary, with an amplifier (e.g. SOA) and then switched by a fast optical switch. If an SOA is used, it can be included in the optical circuit.

Preferred for the described method and the apparatus are swept-source laser sources 18, the wavelength characteristic of which is predictable such that the pulse control can be carried out without online monitoring of the laser wavelength, i.e. without wavelength measurement arrangement (k-clock). If this is not possible, a k-clock arrangement, as known in the prior art, is used to control the synchronization of optical switches.

J. Kühn et al., Optics Express Vol. 15, No. 12, 2007, “Real-time dual-wavelength digital holographic microscopy with a single hologram acquisition,” describe a method of digital multi-frequency holography in which two wavelengths lambda-0 and lambda-n are recorded simultaneously in a camera image. The reference waves of the two wavelengths are here radiated in from different directions in such a way that they can be separated from one another during the evaluation by means of suitable lateral filtering. The advantage of this illumination arrangement is that the reference recording is realized exactly at the same time as the measurement recording, whereby movement artifacts are suppressed even better. This can now be realized with the rolling shutter camera, and the fast switching of the lasers for realizing the integration time is achieved by the camera integration. Thus, the laser sources can be implemented more easily. A disadvantage of this arrangement is that in the direction perpendicular to the off-axis detection, the recording has to be scanned 2 times more densely in order to realize the filtering. Effectively half of the camera pixels are lost thereby. In addition, the two reference waves reduce SNR by 3 dB. This embodiment is therefore preferably used for the measurement of very dynamic objects.

If the described method is combined with scattered wave illumination, the evaluation is slightly modified. In this case, it is not sufficient to subtract the phase of the reference measurement, as propagation of the scattered wave into the sample produces phases and amplitude changes that are not location-variant and thus change when the object is displaced in relation to the measurement device. The spatial frequencies introduced by the scattered speckle illumination mix with the non-limited spatial frequency spectrum of the object to be measured. This mixed frequency spectrum is filtered by the filtering which is then implemented by a detection stop. A clear “demixing” of the transmitted, filtered and detected spatial frequency spectrum is then no longer possible from a single wavelength image stack. Therefore, for the further discussion, a distinction is made between two evaluation modes, a simple mode in which exactly one wavelength image stack is evaluated, and an extended evaluation mode in which a plurality of, preferably about 10 to 40, wavelength image stacks are coherently mathematically combined.

In the simple embodiment, the evaluation does not attempt to demix the sample spatial frequency spectrum which has been mixed with the scattered illumination angle spectrum. Rather, the final image represents the mixture of detection speckles, which are created by backscattering in the object, with the illumination speckles. In order for this method of reconstruction to not cause any artifacts, it is important that during the recording of all images that are to be coherently mathematically combined, the illumination speckles differ laterally and axially from one another and from the object by significantly less than one grain of speckle. For simple evaluation, provision is therefore made for the diffusing plate to be imaged into the middle object plane, so that the speckle patterns of the different wavelengths are also approximately the same except for a wavelength-dependent propagation phase that is characteristic of the method. The depth range that is thereby achievable is scaled with lambda2/(nA²*delta-lambda).

In addition, the images should be recorded as quickly as possible. If the recordings are displaced only by a fixed point, as is the case, for example, with recordings of a human retina that is fixed to a fixation light, it is alternatively possible to select the illumination aperture to be so small that the resulting illumination speckles are larger than the displacement amplitude.

A third option is to select the illumination aperture to be significantly smaller (<3 times) than the detection aperture. In this case, only minor artifacts are produced, which result in higher, but still acceptable image noise. In this case, however, the illumination wave is usually so severely restricted in its etendue that the usable light limit values are only slightly larger than those of single-mode illumination.

If fast movement artifacts with large amplitude and high demands in terms of the lateral resolution and image quality to be achieved occur or if very deep object structures are to be measured that are deeper than the depth range described in the previous section, then an extended evaluation is preferably carried out. A plurality of independent wavelength image stacks are recorded and coherently mathematically combined. There are three variants of this extended evaluation that also have different properties. In some of these variants, the lateral resolution can also be doubled and multiple scattering can be suppressed in the recordings.

For the extended evaluation described below, it is preferred to image the diffusing plate 39 into the object plane, but it is also possible for some of the variants, for example, to place the diffusing plate close to the pupil. This is important in particular in lens-free design variants, as are known in the prior art. The specific problem with this type of evaluation is that all recordings must be mathematically combined coherently with one another. By recording a plurality of image stacks even with cameras of high lateral resolution, phase-fixed image recording is realized, as will be explained, even over several seconds.

FIG. 2 shows a light source 19 with only one laser 42 for the subsequent first and second illumination variants.

The first illumination variant has already been mentioned. There is only one tuned laser source 42, with tuning rates in the order of the frame rate of the camera 22. Owing to the fast circuit 40 of the source 19 synchronized with the recording by the camera 22, the double images with the wavelengths (lambda-0, lambda-n) are suitably cut out and detected. In this case, only a source 42, an optional splitter 44 for an optional k-clock 48 (see above), and a fast switch 46 are required. The disadvantage of this embodiment is that the temporal distance between lambda-0 and lambda-n cannot be chosen as short as desired, since it is limited by the tuning time of the laser 42. In addition, the integration time for a single image lambda-i is limited in this type of detection, since the laser 42 is continuously tuned and only a specific wavelength drift can be tolerated during the integration time.

The second illumination variant differs from the first only by way of different laser control. In this case, the laser 42 is not continuously tuned, but switched between fixed wavelengths. At each new wavelength, the laser requires a specific settling time of about 1 to 100 ms in order to return to stable long-coherence operation. In this case, the integration interval 4.1 etc. can be selected to be significantly longer, so that it is limited only by the movement artifacts. However, the minimum temporal distance within the double image corresponds here to the settling time. This solution is therefore particularly preferred for laser systems that settle into a stable mode in less than approximately 10 ms. During the settling time, the laser is blocked by the switch 46 so as not to falsify the camera signals.

A third illumination variant according to FIG. 3 is carried out with two lasers 42, 50, a fixed-frequency laser 50 with the wavelength lambda-0, and a second tunable laser 42. The tunable laser 42 is either switched between discrete wavelengths lambda-i as in FIG. 2 or is slowly tuned continuously. Slowly means in this case, for example, that if 400 wavelengths lambda-i are used, the laser 42 is tuned once in about 11 seconds, in order to be able to record 400 double images lambda-0, lambda-i with the camera during this time. In this case, a fast optical switch 52 takes over the combining of the two laser beams and additionally the fast optical switching. This variant is technically the most complex, but allows very fast double images and allows long integration intervals. In this constellation, it is particularly preferred if an SOA/optical post-amplifier 54 is implemented downstream of the switch 52 in order to be able to amplify both lasers 42, 50. This variant can also be preferably combined with the described simultaneous 2-reference recording.

In this case, an intensity divider is used instead of the optical switch 52, and the exposure is realized by the integration interval of the camera. The reference waves for the k-clock 48 are divided in the divider 44a, 44b before the combining. As a result, the additional effort is limited to the implementation of the second fixed-frequency laser 50. However, since most OCT recordings only require coherence lengths in the 10 mm range, technically simple reference laser diodes can be used.

If the recording method shown is used for recordings of the cornea and the anterior chamber 16 of the human eye 14, the number of images to be recorded for specific applications can be minimized by means of specific one-beam geometries. Various one-beam geometries with their advantages and disadvantages are to be described below.

The advantage of the OCT system described here is a microscopic resolution in the micrometer range over tissue depths of several millimeters. Parameterization allows the depth of field of the system and the depth resolution achieved by the coherence measurement to be set independently of one another. However, it is important for fully coherent 3D reconstruction that the coherence depth resolution achieved is chosen to be better than or equal to the depth of field of the system.

If the microscopic resolution should only be achieved in the corneal tissue 60, the illumination radiation should preferably be radiated in in such a way that a curved zero-delay surface is formed near the corneal front. The shape and position of the zero delay is equally affected by the illumination wave and backscattered detection wave. To create a zero delay surface on the corneal front, the Fermat principle can be applied. If the corneal front is imagined to be mirrored and the illumination is realized in such a way that it is imaged into the detection by the corneal reflection, the zero delay is set by a length adjustment of the reference arm to the corneal surface. For this, 3 geometries are conceivable, as shown in FIG. 4A-C. Of course, mixed forms can also be realized, however. Detection is accomplished with a specific numerical aperture. In FIG. 4A-C, however, only the chief rays are shown in simplified form. The figure shows beam geometries for zero delay on the corneal front 61 (illumination B dashed, detection M drawn through), the average radius of curvature of the cornea 60 is 8 mm. In FIG. 4A, the detection is targeted at about 4 mm behind the corneal vertex 62. In FIG. 4B, the illumination is targeted at about 4 mm behind the corneal vertex 62. In FIG. 4C, both are targeted at about 8 mm behind the corneal vertex 62.

The advantage of one of the beam guidances according to FIG. 4 is that with a measurable depth range of about 700 μm in the tissue or about 1 mm in air, the entire cornea 60 can be recorded with high resolution. The disadvantage of this arrangement is that the very bright corneal front reflex can override the dynamics of the recording. In addition, larger optical diameters are required to achieve beam guidance.

FIG. 5 shows improved illumination with a parallel beam and telecentric detection. It is better suited for recordings of the entire anterior chamber and for recordings of smaller subfields. In addition, the angles of incidence can be selected more flexibly for recordings of Schlemm's canal, for example, in order to improve imaging by minimizing scleral paths. For this purpose, it is particularly preferred if the measurement device also comprises, in addition to x, y and z adjustment via a cross stage used in ophthalmological devices, a swivel apparatus (possibly tilting apparatus), as are known in fundus cameras and slit lamps.

It is preferable to arrange the optical switch upstream of the split into the signal illumination wave and the reference wave in order to also switch the reference wave and thus minimize the DC component in the camera recordings.

The methods developed in the prior art for movement compensation attempt to compensate the movements only within one wavelength image stack. To do this, an attempt is made in a wavelength scan to distinguish between signal changes caused by the change in the wavelength and signal changes caused by movement artifacts, and then to compensate for the movement artifacts. This has three applicative limitations that do not make it possible to transfer this to the problem described here. The method is suitable only for correcting minor movements within the recording of the image stack. The coherence time of a few milliseconds can thus be extended by a factor of about 10. In order to also achieve the extremely fast frame rates of the camera required for this, high-speed cameras must be used, which have a reduced resolution of only up to about 1 Mpixel. In addition, there must be sufficient lateral and axial structures in the objects to be recorded to allow estimation of the movement artifacts. This is more difficult especially because of the lower number of pixels than for high-resolution camera sensors used here. In summary, the prior art methods estimate the movements from fast wavelength image sequences, while the method and the apparatus presented here estimates the movements even from single images.

Due to the movement artifacts that occur both in the recording of the image stacks and between the image stacks, the images of the same wavelength in the different stacks have a different random displacement of the illumination speckle pattern in relation to the object to be measured, which should be larger than a speckle grain.

In the following text, the particularly preferred first subvariant of the extended evaluation will be described first and then only the differences of the other two possible subvariants are discussed.

For the extended evaluation, the movements between the image pairs are estimated in a first step by comparing the recording with the reference wavelength and the recording of the second wavelength recorded within a short period of time without movements is thus digitally corrected, so that all the recordings of the plurality of image stacks are then coherent with one another.

As a second step, the images of the different stacks are rearranged and a resolution-enhanced image is reconstructed here from all the images of the respectively identical wavelength, which image also comprises suppressed multiple scattering depending on the number of mathematically combined stacks. To this end, the calculated signal field strengths of the various recordings are simply added together. Owing to the phase correction in the first step the single-scattered signal components are constructively superimposed here, while the multiple-scattered signals are superimposed in a phase-uncorrelated manner and thus partly removed from the signals by averaging.

The third step is then again identical to the known holoscopic reconstruction, and the resulting resolution-enhanced images of the different wavelengths are converted into a depth-resolved final image by a Fourier transform. In the prior art, there are also more complicated 3D evaluation methods which can be used as an alternative, but are referred to here below in simplified terms as Fourier transform.

For the particularly important first step of the evaluation, the reference wavelength image is extracted from each recorded image pair and compared with a fixed comparison image, e.g. the first reference wavelength image. The two images are cross-correlated laterally with one another and then the lateral displacement is corrected. The same displacement is also corrected in the associated second wavelength image. The next step is to compare the field strengths of the first and n-th reference images with one another. The phase values will differ from one another for three physically different reasons.

First, because the two reference images may have changed due to axial movements in the absolute phase. These changes can result in a constant global phase, but in most applications higher lateral orders such as tilt angles (phase ramps) and defocus (square lateral phase-type distributions) will also occur and need to be corrected. However, these phase functions are very strongly correlated locally, with the result that the phase change does not have to be estimated individually for each pixel, but a parameterized phase function with a few, up to a few 10, parameters can be fitted.

Secondly, the scattered illumination wave with its typical speckle pattern in each speckle grain will cause a random initial phase. In addition, the excitation field strength magnitude will vary laterally and axially across the object due to this speckle. These speckle amplitudes and phases are assumed to be known. They can be derived from the design of the diffusing plate or can be measured by a calibration measurement with a mirror as the object in a plane and can be transferred/converted from there to all points of the illuminated object using the methods of digital propagation. The mirror/concave mirror is designed here in such a way that it images the exit pupil of the scattered illumination into the entrance pupil of the detection optical unit. If the beam path is telecentric on the object side, plane mirrors are used, otherwise concave mirrors are used. The measured field distribution of the scattered excitation wave is hereinafter referred to as calibration measurement and is recorded individually for each wavelength. The measured signal field strength image is divided in a complex manner by the so-known excitation field strength/calibration measurement in order to compensate for this influence of the speckle illumination. Both variables are determined in the conjugated image plane/zero delay and mathematically combined. Since this division can produce double spatial frequencies, the signal field strength image and calibration image are interpolated to double the pixel density prior to this operation.

The third effect is caused by an interaction of the illumination spatial frequencies with the spatial frequencies of the backscatter coefficient of the object. Thus, the amplitude and phase values in an object point change depending on the gradient of the excitation field strength at that point, which will be different for each position of the illumination speckle pattern relative to the sample.

If, for example, an illumination speckle pattern comprising the same angular aperture as the detection is used for illumination, the double spatial frequency values of the object, which would be calculated solely from the diffraction-limited detection, are accessible owing to the structured speckle illumination. However, only the single spatial frequency spectrum is transmitted by the detection. For this reason, only a quarter of the spatial frequency information in an image is transmitted in this example, and it is not possible in principle to determine this phase and amplitude effect from a single recording. In the later mathematical combination of sufficiently many images of the same wavelength but different illumination speckle displacements, all spatial frequencies up to twice the detection limit frequency can then be unambiguously reconstructed. To this end, however, coherent mathematical combination is absolutely necessary, for which the displacement phases must be determined and corrected beforehand. In this respect, this effect cannot be compensated for here when determining the phase shift of the single images and its effects must be managed. After correction of the detected signal field strength images by the excitation field strengths, the effect thus described leads to a phase decorrelation of approximately 75% in this example. To nevertheless be able to estimate the phase position of the reference images in relation to one another, it is necessary to average laterally over a plurality of (many) pixels. The global phase can be determined by averaging all pixel phases. For practical application, however, the described approach with the fitting of a parameterized phase function with significantly fewer parameters than detection pixels is the clearly preferred option. Once the phases have been determined in this way, the phases are converted from the reference image wavelength Phi-ref to that of the measurement image wavelength Phi-mess (Phi-mess=Phi-ref*lamda-mess/lambda-ref) and corrected in these measurement images with variable wavelength.

The last two steps of the evaluation, the coherent summing of the field strengths of all the images of the same wavelength after the correction described and the subsequent Fourier transform over the different wavelengths, can also be performed in the reverse order due to the linearity of the Fourier transform. To this end, each corrected image stack is transformed individually by a Fourier transform and then the complex result fields are added up coherently.

The first subvariant of the extended evaluation thus altogether has the following properties: The diffusing plate does not necessarily have to be imaged into the object plane, and so this variant is also suitable for lens-free systems. However, the excitation speckle distribution must be known by the calibration measurement. The result image will have a double lateral resolution depending on the choice of illumination aperture. Depending on the number of reconstructed image stacks, the multiple scattering in the result image is suppressed because it is partially removed by averaging. However, the result images will be completely speckled. In order to partially de-speckle the images, 4 pixels in the result image can be averaged incoherently, as a result of which the lateral resolution is reduced to the normal detection resolution, however.

In the second subvariant of the extended evaluation, the signal images are also first divided by the calibration measurement, then follows compensation of the lateral displacements and the axial phases owing to movement artifacts. However, for each image stack first the Fourier transform over the wavelengths is then calculated. However, the resulting depth-resolved images of the different image stacks are then added incoherently, i.e. the amounts of the function are calculated and added together. As a result, a final image with normal lateral resolution diffraction-limited by the detection is obtained, in which the multiple scattering is not suppressed, but which can also be completely de-speckled depending on the number of mathematically combined stacks. This de-speckling refers to both the single-scattered signal components and the multiple-scattered signal components, and so the final images will look very smooth/low-noise. It is then possible to virtually suppress the multiple scattering visible in the image by increasing the displayed image contrast. For this variant, the diffusing plate does not have to be imaged into the sample, making it also suitable for lens-free systems.

In the third subvariant of the extended evaluation, the signal image is not divided by the calibration measurement. However, the lateral displacements and the axial phase changes resulting from the movement are corrected as described in the other subvariants. As with the first subvariant, the images are first added coherently and then the Fourier transform over the wavelengths is calculated or else in reverse order. This variant of the evaluation only works if, as with the simple evaluation, the diffusing plate is sharply imaged into the sample and the maximum depth range shown is maintained. In this case, the initial phases of the scattered excitation wave in the zero delay are not known, but they are approximately the same at all wavelengths and thus stand out in the evaluation. Therefore, in this variant, the single-scattered signal components are superposed constructively and the multiple-scattered signal components are superposed in uncorrelated fashion, as a result of which the images will show suppressed multiple scattering. However, since the intensity fluctuations of the scattered illumination wave, which also affect the transmission of the spatial frequencies, are not corrected, the higher orders of diffraction will stand out and the result image will have only the simple resolution diffraction-limited by the detection. However, the result images obtained are at least partially de-speckled. The basics of the evaluation according to the third subvariant are also described in DE 10 2018 130 396 A1.

A feature of the described arrangement is the very high degree of parallelization with a resolution of many megapixels. As a result, the excitation intensity per pixel is very low, so either very long integration times follow or very powerful lasers have to be used. In the following text, therefore, special refinements of the light source system will be described, which are preferred for this application range.

The arrangements shown show only the aspects of the absolute beam paths traveled and no imaging performance and image field positions. The correction of such large image fields of up to about 12 mm for numerical apertures of 0.2 and larger with simultaneous diffraction-limited resolution is optically very difficult to achieve, especially even for image field planes that are convexly curved. However, a major advantage of the fully coherent wide-field OCT is that known image fields can be removed by calculation from the data during evaluation in that the detected light wave fields are propagated numerically to the pupil, the phase function of the aberrations is corrected there, and the wave is then propagated back numerically into the field plane. This method is known in the prior art. In this case, it is moreover particularly preferred that the optical unit is corrected to maximum sharpness in the peripheries of the image field at the expense of the correction in the center of the image field (specifically in the image field curvature) in order to minimize the periphery effects/overlap region during numerical propagation.

1. A wide-field swept-source OCT method for imaging a moving object (14), in particular the anterior chamber (16) of the human eye, wherein the method comprises the following steps:
providing illumination radiation (B), which is tuned in the wavelength and comprises individual illumination pulses (10.L0, 10.L1) of different centroid wavelength, wherein the illumination pulses (10.L0, 10,L1, 10.L2) are provided as a series of illumination pulse pairs (10.L0, 10L1; 10.L0, 10.L2), each consisting of a first illumination pulse (10.L0) and a second illumination pulse (10.L1, 10.L2), wherein in the illumination pulse pairs (10.L0, 10L1; 10.L0, 10.L2) the centroid wavelength of the first illumination pulse (10.L0) differs from the centroid wavelength of the second illumination pulse (10L1, 10.L2), and in a plurality of the illumination pulse pairs (10.L0, 10L1; 10.L0, 10.L2) at least one centroid wavelength of one of the illumination pulses (10.L0) of a preceding one of the illumination pulse pairs (10.L0, 10.L1; 10.L0, 10.L2) is repeated,
illuminating the object (14) with the illumination radiation (B) and imaging the illuminated object (14) onto a 2D detector (22),
operating the detector (22) according to an image recording cycle comprising a sequence (2) of exposure intervals (4.1, 4.2, 4.3, 4.4) and readout intervals (6.1, 6.2, 6.3),
synchronizing the emission of the illumination pulses (10.L0, 10,L1, 10.L.2) and the operation of the detector (22) in such a way that the illumination pulse pairs (10.L0, 10L1; 10.L0, 10.L2) are grouped around every second one (6.1, 6.3) of the readout intervals (6.1, 6.2, 6.3) of the sequence (2) and, for each illumination pulse pair (10.L0, 10L1; 10.L0, 10.L2), the first illumination pulse (10.L0) is emitted during a last third of one of the exposure intervals (4.1, 4.2, 4.3, 4.4) and the second illumination pulse (10L1; 10.L2) is emitted during a first third of the next of the exposure intervals (4.1, 4.2, 4.3, 4.4), and
reading image data of each exposure interval (4.1, 4.2, 4.3, 4.4) of the detector (22) and assigning the image data to the centroid wavelengths of the illumination pulse (10.L0, 10,L1, 10.L.2) emitted in the respective exposure interval, wherein image pairs consisting of single images are generated according to the illumination pulse pairs (10.L0, 10L1; 10.L0, 10.L2),
determining changes in the image pairs between the single images to which the same centroid wavelength is assigned, and
evaluating the image data and using the changes to correct movements of the object (14) in the image data.

2. The method as claimed in claim 1, wherein in a plurality of illumination pulse pairs (10.L0, 10L1; 10.L0, 10.L2) exactly one centroid wavelength of one of the illumination pulses (10.L0) of the immediately preceding one of the illumination pulse pairs (10.L0, 10L1; 10.L0, 10.L2) is repeated.

3. The method as claimed in claim 2, wherein the same centroid wavelength is repeated as the reference centroid wavelength.

4. The method as claimed in any of the above claims, wherein a temporal distance (12) of the illumination pulses of the illumination pulse pairs (10.L0, 10L1; 10.L0, 10.L2) is selected such that the single images of each image pair differ in location from the object (14) by less than one speckle grain of the illumination.

5. The method as claimed in any of the above claims, wherein a movement during the imaging of the object is realized by a motorically moved object carrier.

6. The method as claimed in any of the above claims, wherein a non-living in-vitro object is imaged and illuminated for this purpose with illumination radiation (B) formed as scattering wave illumination, wherein a plurality of wavelength image stacks are recorded and a displacement between illumination speckles and the object is achieved by a motorized movement of the object.

7. The method as claimed in any of the above claims, wherein this motorized movement of the object is carried out only between the image stacks and not within the image stacks.

8. The method as claimed in any of the above claims, wherein the measurement radiation is superimposed with reference radiation on the detector (22), wherein different reference radiation directions are provided for the single images of each image pair, and the two single images are separated in an image evaluation based on the reference radiation directions.

9. A wide-field swept-source OCT for imaging a moving object (14), in particular the anterior chamber (16) of the human eye, wherein the OCT (12) comprises:
a radiation source (18), which emits illumination radiation (B) which is tuned in the wavelength and comprises individual illumination pulses (10.L0, 10.L1) of different centroid wavelength, wherein the illumination pulses (10.L0, 10,L1, 10.L2) are emitted as a series of illumination pulse pairs (10.L0, 10L1; 10.L0, 10.L2), each consisting of a first illumination pulse (10.L0) and a second illumination pulse (10.L1, 10.L2), wherein in the illumination pulse pairs (10.L0, 10L1; 10.L0, 10.L2) the centroid wavelength of the first illumination pulse (10.L0) differs from the centroid wavelength of the second illumination pulse (10L1, 10.L2), and in a plurality of the illumination pulse pairs (10.L0, 10L1; 10.L0, 10.L2) at least one centroid wavelength of one of the illumination pulses (10.L0) of a preceding one of the illumination pulse pairs (10.L0, 10L1; 10.L0, 10.L2) is repeated,
a 2D detector (22), which performs an image recording cycle comprising a sequence (2) of exposure intervals (4.1, 4.2, 4.3, 4.4) and readout intervals (6.1, 6.2, 6.3),
a beam path (24) for illuminating the object (14) with the illumination radiation (B) and for imaging the illuminated object (14) onto the 2D detector (22), and
a control device (C), which controls the 2D detector (22) and the radiation source (18) and is configured to synchronize the radiation source (18) and the 2D detector (22) in such a way that the illumination pulse pairs (10.L0, 10L1; 10.L0, 10.L2) are grouped around every second one (6.1, 6.3) of the readout intervals (6.1, 6.2, 6.3) of the sequence (2) and, for each illumination pulse pair (10.L0, 10L1; 10.L0, 10.L2), the first illumination pulse (10.L0) is emitted during a last third of one of the exposure intervals (4.1, 4.2, 4.3, 4.4) and the second illumination pulse (10L1; 10.L2) is emitted during a first third of the next of the exposure intervals (4.1, 4.2, 4.3, 4.4),
wherein the control device (C) is further configured
for reading image data for each exposure interval (4.1, 4.2, 4.3, 4.4) of the detector (22) and for assigning the image data to the centroid wavelengths of the illumination pulse (10.L0, 10,L1, 10.L.2) emitted in the respective exposure interval, and for generating image pairs according to the illumination pulse pairs (10.L0, 10L1; 10.L0, 10.L2),
for determining changes between the image data of the illumination pulses (10.L0) with the same centroid wavelength repeated across the illumination pulse pairs (10.L0, 10L1; 10.L0, 10.L2), and
for evaluating the image data and for using the changes to correct movements of the object (14) in the image data.

10. The OCT as claimed in claim 9, wherein the radiation source (18) comprises a swept-source laser (42), which has a tuning repetition rate which is not lower than a frame rate of the detector (22) defined by the duration of the exposure interval (4.1, 4.2, 4.3, 4.4) and readout interval (6.1, 6.2, 6.3).

11. The OCT as claimed in claim 9 or 10, wherein the radiation source (18) comprises an optical switch (46) or switch (52) controlled by the control device (C) for synchronization.

12. The OCT as claimed in any of claims 9 to 11, wherein a coherence depth resolution is better than or equal to a depth of field defined by the beam path.
